(19) United States
(12) Patent Application Publication
(10) Pub. No.: US 2024/0393316 A1
(43) Pub. Date: Nov. 28, 2024

(54) Improving Production Safety and Efficiency by Early Detection of Sick and/or Dead Individuals and Accurate Determination of Time of Death Using UV Light and/or Visible Light

(71) Applicant: KONRAD WLODARCZYK, Maslomiaca (PL)

(72) Inventor: KONRAD WLODARCZYK, Maslomiaca (PL)

(21) Appl. No.: 18/673,314

(22) Filed: May 24, 2024

Related U.S. Application Data
(60) Provisional application No. 63/468,555, filed on May 24, 2023.

(51) Int. Cl. G01N 33/483; A01M 1/04

(52) U.S. Cl. CPC G01N 33/4833; A01M 1/04

(57) ABSTRACT

Combining UV and/or visible light, a conveyor, a computer and photodetectors is an efficient and cost-effective way to determine if any reared and breeding insects are sick and/or dead. A pool of insects placed on a conveyor allows sick and/or dead insects that are present in the pool to be detected because they move into a beam of UV and/or visible light, wherein they emit light (e.g., fluorescent light), which can be detected by photodetectors. The computer that may be operationally attached to the UV and/or visible light, the photodetectors, and the conveyor may be able to ascertain an exact position where the one or more sick and/or dead insects is/are.

This application claims priority under 35 USC 119 (e) to U.S. Provisional Application No. 63/468,555 filed May 24, 2023, the entire contents of which are hereby incorporated by reference in its entirety.

### FIELD OF THE INVENTION

The present invention relates to systems and methods for ascertaining if an insect is sick dying, dead or healthy alive. The systems and methods of the present invention use ultraviolet light, and in some instances, either UV or visible spectrum light to irradiate the insects, wherein dead or terminally ill insects emit the fluorescence radiation that live and healthy insects do not. The systems and methods of the present invention are superior to any methods of the prior art in that they rely on photodetectors rather than simply by visual inspection of the insects.

### BACKGROUND OF THE INVENTION

Industrial-scale insect rearing and breeding is one of the fastest developing methods for obtaining alternative animal protein. For its development, however, it is essential to reduce the high costs of manual handling in breeding, particularly to be able to identify dead individuals. Automation and robotization of insect rearing, breeding and processing processes are the key to achieving profitability in order to compete with existing methods for obtaining protein to be used for food and feed. The methods of the prior art tend to be very destructive to our planet's ecosystem. The use of machine learning algorithms and the application of artificial intelligence to control and optimize production processes in insect rearing, breeding and processing requires the provision of adequate quantity and quality of data from vision systems, with the data replacing the caretaker's organoleptic assessment during manual handling. For this purpose, it is possible to obtain data of insects generated under ultraviolet light.

The phenomenon of luminescence of organisms has been known to mankind for hundreds of years. Early in antiquity, Aristotle and Pliny the Elder wondered about the source of light that could be observed in some dead fish and in decayed wood [Biron K: Fireflies, dead fish and a glowing bunny: a primer on bioluminescence. BioTeach J., 2003; 1:19-26].

The phenomenon of bioluminescence in living insects is known and is caused by luciferase. An insect that is known to have luciferase is the beetle of the species Photinus pyralispyralis [Luker K. E., Luker G. D.: Applications of bioluminescence imaging to antiviral research and therapy: multiple luciferase enzymes and quantitation. Antiviral Res., 2008; 78:179187]. Its presence has also been demonstrated in some species from the insect families Lampiridae, Elateridae or Phengodidae [Stevani C. V., Oliveira A. G., Mendes L. F., Ventura F. F., Waldenmaier H. E., Carvalho R. P., Pereira T. A.: Current status of research on fungal bioluminescence: biochemistry and prospects for ecotoxicological application. Photochem. Photobiol., 2013; 89:1318-1326].

The phenomenon of fluorescence of certain organisms, which is the emission of light under the influence of a stimulating agent, is also known. An example thereof may be the fluorescence of scorpions under the effect of ultraviolet radiation. In this case, the effect is the emission of light in the green-blue color region. Beta-carboline and 7-hydroxy-4-methylcoumarin found in the outer layer of chitin in these organisms are responsible for its fluorescence.

Insects in industrial production are usually separated according to their developmental stage and size and are kept in high-density mono groups. In the event of unfavorable environmental conditions or the presence of a pathogen, mortality occurs in waves, starting with individuals and then spreading to increasing numbers thereof until the whole group, i.e., all individuals from one breeding container, become extinct. With a sufficiently high ratio of dead individuals, even when the lethal factor is no longer present, the whole breeding group dies out. Therefore, sufficiently early detection of the first sick or dead individual is crucial, thereby providing the opportunity for an early response to identify and correct adverse factors causing mortality. Bringing the process to a halt and removing the sick and/or dead individual (or individuals) often saves the entire production from collapsing completely. Moreover, preventing the sick and dead individual (individuals) from staying during breeding leads to a high-quality product.

Sick or even dead insects in larval form can, although it is extremely complicated, be identified by means of vision systems that detect movement. However, in the case of species or life stages where movement is negligible (e.g. pupae), detection of terminally ill or dead individuals in this way is not possible or takes too long to be applied effectively in a continuous production process. The only way to do this is to visually assess the changing appearance of the dead individual, e.g., changes in color and/or shape, as influenced by decomposition/desiccation processes. However, it takes as long as seven days after death for the first signs of these processes to appear, depending on the species and environmental conditions, by which time the whole group is usually infected and it is too late to implement remedial processes.

It is with these limitations of identifying individual sick or dead insects that the present invention was developed.

### BRIEF SUMMARY OF THE INVENTION

The present invention relates to systems and methods for ascertaining if an insect is terminally ill, dead or alive. The systems and methods of the present invention use ultraviolet light and/or visible light to irradiate the insects, wherein ill and dead insects emit the fluorescent radiation that live insects do not. The systems and methods of the present invention are superior to any methods of the prior art in that they rely on photodetectors rather than simply by visual inspection of the insects.

### BRIEF DESCRIPTION OF THE SEVERAL VIEWS OF THE DRAWING

FIG. 1 shows a schematic representation of one embodiment of the present invention that uses a UV light and/or a visible light source, a conveyor, and photodetectors to determine if dead insects are present.

FIG. 2 shows a graph showing the emission (blue) and excitation (orange) spectra of examined pupae.

FIG. 3 shows dead larvae and pupae after a night in the fridge at 7° C., wherein there is no visible impact on the fluorescent effect.

FIG. 4 shows dead larvae and pupae after a night in the freezer −20° C., wherein there is no visible impact on the fluorescent effect.

FIG. 5 shows dead larvae and pupae after drying in 80° C. after 3 h, wherein the fluorescent effect is a bit weaker, but nevertheless, still present.

FIG. 6 shows dead larvae and pupae from FIG. 5 with fluorescent effect in the top image whereas the dead larvae and pupae that were deactivated and dried (underwent a processing step) with a hot air showed no fluorescent effect.

FIG. 7 shows dead larvae and pupae before pouring boiling water on to them.

FIG. 8 shows dead larvae and pupae after pouring boiling water, and there is no visible impact on the fluorescent effect.

FIG. 9 shows dead larvae and pupae after pouring boiling water and leaving for a night and the fluorescent effect is still strong.

FIG. 10 shows dead larvae and pupae after pouring cold water and leaving for a night.

FIG. 11 shows a comparison of water from larvae and pupae bath wherein the top image shows them after boiling, and the bottom image shows them after being exposed to cold and both show a strong fluorescent effect.

FIG. 12 shows larvae and pupae after drying in a microwave for 10 min and the fluorescent effect is weaker, but still present.

FIG. 13 shows on the right, water from larvae and pupae bath (FIG. 11) and on the left, it shows water with larvae that was intentionally deactivated (killed) and dried wherein there is no fluorescent effect.

FIG. 14 shows a detail image of pupae.

FIG. 15 shows larvae and pupae in a cup wherein there is a fluorescent effect present whereas in the background, a large amount of larvae are shown after deactivation and drying with no fluorescent effect.

FIG. 16 shows dead larvae and pupae under white light.

FIG. 17 shows dead larvae and pupae in the fridge at 6° C. for two weeks and although the fluorescent effect is weaker, it is still present.

FIG. 18 shows dead larvae and pupae in the fridge stored at 6° C. for two weeks in a plastic bag under white light.

FIG. 19 shows dead larvae and pupae in the fridge stored at 6° C. for two weeks in a plastic bag wherein the fluorescent effect is present.

FIGS. 20A and 20B show dead larvae and pupae in the freezer stored at −20° C. for two weeks and there is no visible difference under white light (prior to measuring for fluorescence).

FIG. 21A and FIG. 21B shows dead larvae and pupae in the freezer stored at −20° C. at time 0 (21A) and for two weeks (21B) and there is no visible difference in the fluorescence present.

FIG. 22 shows terminally ill larvae that died within 12 hours wherein the terminally ill larvae show a fluorescent effect.

FIGS. 23A and 23B show the solution obtained from the dead larvae/pupae stored in the fridge at 6° C. at time zero and at two weeks under white light (23A) and after fluorescing (23B), and in both instances there is no appreciable difference.

### DETAILED DESCRIPTION OF THE INVENTION

The present invention relates to systems and methods for ascertaining if an insect is terminally ill, dead or healthy alive. The systems and methods of the present invention use ultraviolet and/or visible light to irradiate the insects, wherein dead insects or insects that are terminally sick (shortly before their death) emit the fluorescent radiation that live and healthy insects do not. The systems and methods of the present invention are superior to any methods of the prior art in that they rely on photodetectors rather than by visual inspection of the insects.

Dead or sick insects in larval form can, although it is extremely complicated, be identified by means of vision systems that detect movement. However, in the case of species or life stages where movement is negligible (e.g., in pupae), detection of dead and/or terminally ill individuals in this way is not possible or takes too long to be applied effectively in a continuous production process. Prior to the present invention, the only way to do this is/was to visually assess the changing appearance of the sick and dead individual, e.g., changes in color and/or shape, as influenced by decomposition/desiccation processes. However, it takes as long as seven days after death for the first signs of these processes to appear, depending on the species and environmental conditions, by which time the whole group is usually infected by a lethal factor and it is too late to implement remedial processes. Thus, in an embodiment, the present invention is able to more rapidly identify the dead insects prior to the insects infecting the whole group of insects.

In an embodiment, the present invention relates to developing a method for detecting sick or dead insects that allows for the earliest possible detection of lethally ill and dead individuals before lesions visible under white light emerge. Indeed, it unexpectedly turned out that the use of ultraviolet and/or visible radiation of different wavelengths allows for precise detection of sick and/or dead insects, days before the emergence of any symptoms allowing the phenomenon to be detected in any other way, despite the fact that the phenomenon of fluorescence of ill/dead insects was not known until now.

Thus, in one embodiment of the invention, the present invention relates to methods for detecting terminally sick and/or dead insects, including fragments thereof, by placing the insects within the range of a UV and/or visible radiation source and within the field of view of photodetectors, and optionally a computer to process results from the photodetection by these photodetectors, the photodetection of the insects provides an accurate means of identifying the sick and/or dead insects a long time (days) prior to other methods (such as their detection by using visible light).

Insects in industrial production are usually separated according to their developmental stage and/or size and are kept in high-density mono groups. In the event of unfavorable environmental conditions or the presence of a pathogen, mortality occurs in waves, starting with individuals and then spreading to increasing numbers thereof until the whole group, i.e., all individuals from one breeding container, becomes extinct. With a sufficiently high ratio of ill and/or dead individuals, even when the lethal factor is no longer present, the whole breeding groups die out. Therefore, sufficiently early detection of the first symptoms of dead individuals is crucial, thereby providing the opportunity for an early response to identify and correct adverse factors causing mortality. Bringing the process to a halt often saves the entire production from collapsing completely. It is also crucial to ensure the safety of production.

In an embodiment, the present invention relates to rearing and breeding insects and irradiating these insects with UV and/or visible light. The terminally ill and dead insects contain molecules that upon an incident beam of UV and/or visible irradiation hitting the insects, the dead insects possess these molecules that in turn are stimulated to undergo an electronic transition, thus emitting electromagnetic radiation that can be detected by the photodetectors. The photodetectors are superior to visual inspection or even differences that can be measured in images by a computer in that photodetectors can be more sensitive and thus, dead insects can not only be detected more accurately, the accuracy of the photodetectors also allows for earlier detection of sick and/or dead insects.

The photodetectors are sensitive enough so that even a single ill or dead insect can be detected by the photodetectors.

In an embodiment, the photodetectors are tuned to a given wavelength or a small range of wavelengths so that only the transmitted wavelength of the molecule that undergoes the electronic transition is detected. For example, if it is known that the molecule undergoes an electronic transition that transmits electromagnetic radiation at 470 nm, the photodetectors can be tuned to this particular wavelength so that only electromagnetic radiation at 470 nm or very near 470 nm cause the photodetector to detect this transmitted radiation. Bandpass filters can be employed to generate monochromatic or a small range of electromagnetic radiation.

In one embodiment, the present invention contemplates using a conveyor belt that has insects undergoing rearing and/or breeding. The insects are passed through a region wherein the insects are irradiated with UV electromagnetic radiation (for example at 395 nm) and/or visible irradiation. It should be understood that the UV electromagnetic radiation may be between 315 to 400 nm, or alternatively between 350 and 400 nm. Or the electromagnetic radiation may be between 300 to 450 nm. In a variation, and for example, an industrial light source may be used that provides UV irradiation at 365 nm and/or 395 nm, wherein both of these values fall within the UV spectral range, but it contemplated that irradiation may also occur in the visible region such as at 410 nm. In some embodiments, illumination based on 410 nm diodes may provide superior results even though the diodes generating the light may be more expensive. In a variation, the electromagnetic radiation may be between 365 and 395 nm, or alternatively, about 365 nm, or alternatively at about 395 nm, so long as the molecules that are present in the dead pupae are able to undergo the electronic transition that can be detected by the photodetectors.

After irradiating the insects with UV light, using photodetectors may also prove to be superior to using strictly visible light and images to determine the presence of one or more sick or dead insects because images that are loaded into the computer often need to have pre-processing and processing that is not required when photodetectors are used. This makes the system simple and robust, which is an important advantage for industrial use.

A photodetector is an optoelectronic device that is used to detect incident light or optical power at a given wavelength. The photodetector is able to convert this incident light into an electrical signal. Usually, the signal is proportional to the incident optical power of the electromagnetic radiation that triggers the photodetector. However, it should be understood that a single photodetector that undergoes a transition may be sufficient to trigger a response that notifies the user to alert one that a difference is present (e.g., a dead insect maybe present).

In the photodetector, there is a semiconductor material that is illuminated through photons that have high or equivalent energies to its bandgap. These absorbed photons encourage valence band electrons to move into the conduction band of the semiconductor material, meaning that there are holes in the valence band. The electrons in the conduction band perform as free electrons (holes) that can disperse under the power of an intrinsic or externally applied electric field.

The photo-generated electron-hole pairs because of optical absorption may recombine & re-emit light unless subjected to an electric field-mediated separation to give an increase to a photocurrent. This is a fraction of the photo-generated free charge carriers received at the electrodes of the photodetector arrangement. As discussed previously, the photocurrent magnitude at a specified wavelength is directly proportional to the intensity of incident light, but the photodetectors can be set at a threshold level that allows a signal to be generated with only one or a few photodetectors triggered.

Thus, in an embodiment, there may exist a series of photodetectors that are designed to detect the transmitted radiation from the electronic transition of the molecule that is present in a dead insect. If a threshold level of one or more of the photodetectors undergoes the movement of electrons, the photodetectors may “detect” the molecule's transmitted radiation that has been irradiated with UV and/or visible irradiation light.

In an embodiment, and as discussed above, if the breeding and/or reared insects are present on a conveyor belt, the insects on the belt may be subjected to incident UV irradiation and/or visible irradiation that is pulsed at given intervals. Insects are moving on the conveyor belt, and an encoder is connected to the pulse meter which allows one to ascertain what region of insects are undergoing the pulsed irradiation at a given time. Optionally and/or additionally the encoder can be connected to a computer/PLC, for data collection purposes. The photodetectors may be arranged in a linear manner or in another manner that allows transmitted radiation (if there is any) from the insects (i.e., sick and/or dead insects) to be detected by the photodetectors. The photodetectors, in an embodiment, should be positioned so that they can detect the irradiation that results from ill and dead insects and should be positioned in a manner and connected to a digital circuit so that data is obtained and processed. In an embodiment, an encoder is connected with a digital circuit so that a position of a region of the insects on the conveyor belt that is being irradiated with UV irradiation and/or visible irradiation at a given time is known. If one or more dead insects are present, the photodetectors, which are present at a precise location, will “trigger” due to transmitted radiation from the sick or dead insects. Detections will be reflected on the LED's array, as the precise time/sequence at which the one or more photodetectors were triggered is known. Optionally and/or additionally, a computer can be connected to the digital circuit to acquire data from the encoder and detect signal(s) from photodetectors for data collection purposes.

In an embodiment, there may also be a means of automatically getting rid of the one or more insects that are sick or dead while keeping those that are healthy and alive. For example, there may be a means of separating the one or more sick and/or dead insects from the entire pool of breeding/rearing insects. In an embodiment, the one or more insects may be separated from the other healthy and non-dead insects by simply brushing the insects off of the conveyor belt/out of the tray. In a variation the insects can be separated while free falling from one conveyor to another by blowing it with compressed air and/or by hitting with a micro-actuator. In a variation, the entire tray/container of insects may be removed. Alternatively and/or additionally, there may be “picker” that picks out the dead insect from the pool of insects. In an embodiment, insects that are in close proximity to the sick or dead insect may also be separated from the pool of insects so as to avoid contaminating the entire pool.

It should be noted that sampling given regions of insects should be done so that the entire population/pool of the rearing/breeding insects can be irradiated in a period of time. For example, if one irradiates one tenth of the population with UV light and/or visible irradiation every minute, the other nine tenths of the population should be irradiated systematically and in order so that the entire population is irradiated every ten minutes (i.e., one tenth of the population is irradiated every minute). In an embodiment, the pulses of UV light and/or visible irradiation may occur between every millisecond to 10 seconds, or alternatively, from one millisecond to about 100 milliseconds, meaning that if one tenth of the population is irradiated with UV light and/or visible light, the entire population will be irradiated every 10 seconds (for the one second pulse) to every 10 milliseconds (for a one millisecond pulse). The pulse times can be adjusted to be even faster or slower as needed.

In an embodiment, the insects may be inspected continuously, so the entire population on the belt or/and tray is inspected.

In an embodiment, the insects may be positioned on a circular conveyor belt. In this embodiment, the insects move in a circular direction and the UV and/or visible irradiating electromagnetic radiation and the photodetectors are appropriately positioned so as to sample the insects as they move in the circular direction. In an embodiment, the insects may be positioned on a conveyor belt that is similar to a shaker that moves back and forth so that the insects can be sampled as they move back and forth. No matter the configuration of the conveyor belt, the insects should be positioned so that the entire population of insects can be sampled over a period of time that is not too long. The period of time should not be longer than a period of time that allows one sick or dead insect to contaminate the entire pool (or a region) of insects. The period of time, in one embodiment, should not be so short that great amounts of energy are wasted.

In an embodiment the insects may be separated from the feed and frass when placed on the conveyor belt. In an embodiment, the separated insects may be placed in trays that are in turn placed on a conveyor belt.

In an embodiment, the sampling of the insects (i.e., irradiation with UV light and/or visible light) and the movement of the conveyor belt should be coordinated so that the entire population of insects occurs in a given period of time. The coordinated movement should also be present so that a computer program is able to ascertain the exact location of one or more sick or dead insects (should it occur) when the photodetectors are triggered.

In an embodiment, the conveyor belt may stop when a sick or dead insect is detected and a computer program may alert a person as to the exact location where the sick or dead insect is. This will allow the person to remove the sick or dead insect and/or alternatively to remove a portion of insects, or to remove an entire tray of insects.

In an embodiment, the photodetectors may be connected to a matrix of LEDs that allow one to visualize the one or more sick or dead insects.

In an embodiment, the wavelength of UV irradiation is from 365 nm to 395 nm. In a variation the wavelength irradiation is in the range of 300 nm to 365 nm or 395 nm to 450 nm.

In an embodiment, the radiation wavelength is 410 nm. In a variation the radiation wavelength is in the range of 400 nm to 800 nm (in the visible region).

In an embodiment, the insects are those of the order Coleoptera. In a variation, the insects are those of the species Tenebrio molitor, Alphitobius diaperinuis or Zophobas morio. In a variation, the insects are at different stages of development.

In a variation, the photodetectors may be connected to an image-recording device and the image-recording device may be a digital camera or a digital camcorder. In a variation, the image-recording device is a digital camcorder, smart camcorder, digital camera or smart glasses. In an embodiment, the images may be analyzed by means of a computer equipped with a computer software equipped with machine learning algorithms.

In a variation of the invention, the present invention relates to a system for detecting sick and dead insects comprising a UV and/or visible radiation source and an image-recording device connected to a computer, and an insect tray, the operation of the UV and/or visible radiation source and the image-recording device being synchronized and electronically controlled by means of the computer.

In a variation, the system is provided with a conveyor line for moving the insects, which is electronically controlled by the computer. In a variation, the computer may be equipped with a screen. In a variation, the computer is equipped with computer software equipped with machine learning algorithms.

In a variation, the UV and/or visible radiation source and the image-recording device are integrated into a single unit.

### Example

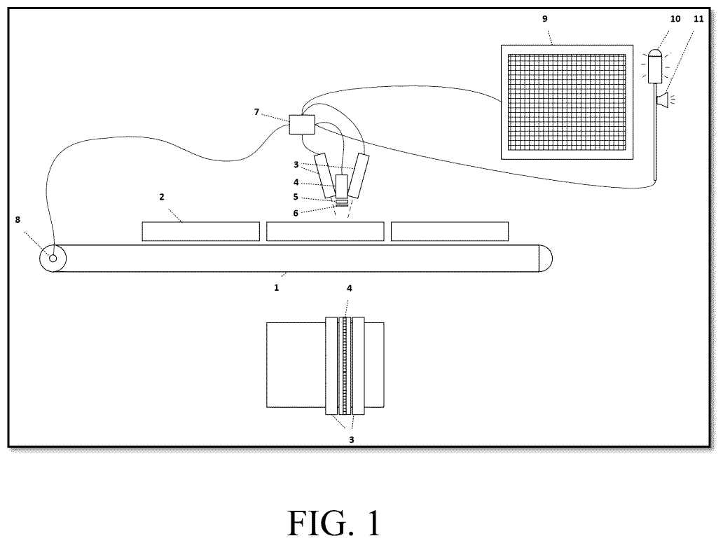

In an example, and as shown in FIG. 1, the present invention may comprise a stand that emits UV irradiation at 365 nm, light source 3 directed at the insects. The photodetectors 4 may be positioned in a row. The photodetectors are equipped with a lens 5 and a bandpass filter 6 that filters 460-490 nm+/−10 nm radiation. The objects/insects are inspected in containers 2 placed on a conveyor 1. An encoder 8 is linked with the conveyor drive and a UV light source 3 so as to emit UV light and give data of the irradiated insects. In an embodiment, a linear speed is available and pulses are initiated using a strobe of UV illumination. A photodetector gives a signal if it receives a certain amount of light going through the bandpass filter. The light is emitted only by dead and/or sick insects (objects) meaning that only dead and sick insects are detected. The photodetectors 4, in an embodiment, are connected with a digital circuit 7 (e.g., a sequential circuit), that processes signals from the row of photodetectors and subsequent signals initiated by the encoder 8 pulses. In an embodiment, the digital circuit is connected with a matrix 9 made of LED's to visualize the results. Alternatively, the photodetectors may trigger a digital circuit that gives a visual 10 and audio 11 signal that alerts a user to one or more dead insects.

As shown in FIG. 1, a pool of insects are placed on a conveyor that moves in a given direction. A UV light source irradiates the pool of insects in a row and the one or more photodetectors are present under insects and are also present in a row. If dead insects are encountered, the one or more photodetectors are activated alerting a user by audio/visual signal and/or a computer or MES or ERP system that can be optionally operationally connected to the digital circuit 7 that one or more dead insects has/have been encountered and identified. In FIG. 1, the conveyor may be a conveyor that moves the insects in a back-and-forth direction to allow scanning of the entire pool of insects in a given period of time. The conveyor and UV light pulses in an embodiment are coordinated in a temporal sense so that one can determine a precise location of the presence of a dead insect. In FIG. 1, a 2D image may be created by the scanning using the UV pulses and detection by the photodetectors allowing a user to visually inspect the 2D image 9 so as to allow the user to identify a precise location where the dead insect (if present) is located.

For example, assuming that a 600 mm width belt/tray is to be inspected, the use of a photodetector allows a user to “see” an area of 4×4 mm. If 150 photodetectors are used with no overlap, this allows one to “see” an area that is 600×4 mm. Accordingly, to scan 1 m, 250 pulses/samples/triggers (1000 mm/4 mm, with no overlap) are needed. In an embodiment, each photodetector is connected to a simple digital circuit, so the photodetector registers a ‘l’ when a dead (or sick) insect is detected, or ‘0’ when there are too few photons to trigger a response in the photodetector. The array of photodetectors can be set so an alarm only triggers when at least two neighboring photodetectors give a triggered signal ‘1’. Because the width of the belt tray is known, the position of the dead (or sick) insect in an x direction can be detected because the signals are available at the time of triggering. In the length dimension (i.e., a y direction), the present invention contemplates including the use of memory (e.g., in a sequential circuit) that will compare the current and a previous (n−1) state allowing one to ascertain immediately a position (and a time) of the dead (or sick) insect(s).

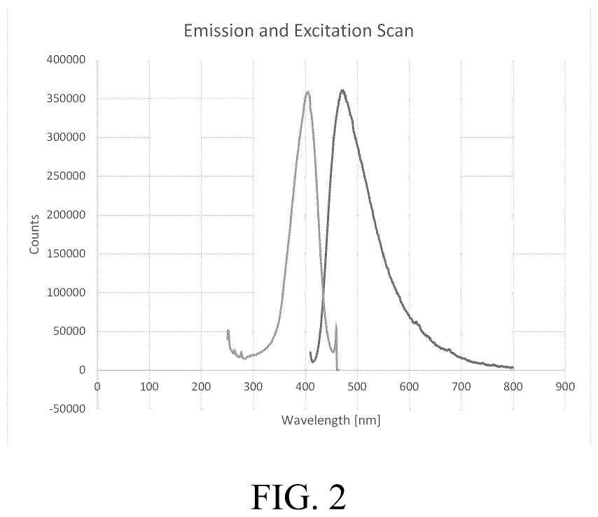

In an embodiment, the present invention relates to dead insects that luminesce (e.g., fluoresce) when irradiated with UV and/or visible light. In an embodiment, the insects may be irradiated with light in the 300-400 nm range (UV) and/or with light in the 400-800 nm range or 400-700 nm range, or in the 400-450 nm range (visible light). It is contemplated that a combination of UV light and visible light may be used in certain circumstances. In an embodiment, the insects may be irradiated with light in a specific wavelength such as at 365 nm, 395 nm (both UV light) or 410 nm (visible). It has been confirmed that the dead insects luminesce (i.e., they give off light by undergoing an electronic transition) rather than merely reflecting the incident light. This is confirmed in FIG. 2 that shows the emission and excitation spectra of pupae. The different wavelengths confirm that an electronic transition is occurring rather than merely reflecting the incident light. One benefit to this fact is that with the appropriate use of filters on the photodetectors, one can be assured that the measured light (the luminescence) comes only from pupae and not from measuring at least a partial contribution from incident light. This means that one can attain very accurate results.

It has been confirmed that sick pupae or larvae emit electromagnetic radiation (fluorescence) when exposed to UV and/or visible light radiation. These individuals die several hours after the phenomenon occurs. This result allows one to potentially isolate the dying pupae or larvae even prior to their death, thereby further reducing the possibility of contamination of the insect pool.

Testing was performed that confirmed that the fluorescence lifetime (FLT) was measured to give a value of 3 ns, which is further confirmation that fluorescence is involved in the electronic transition (as fluorescence ranges from 10⁻⁹ to 10⁻⁶ s). Incident radiation within the range of 300-450 nm will cause excitation of the objects. The excitation peak was measured and found to be 410 nm. The emission peak is seen at 470 nm, which fits within the visible range of 400-800 nm, so it is in the region that is visible to the human eye. Additional testing confirmed that very low temperatures (liquid nitrogen) don't stop the luminescent phenomenon and the present invention contemplates taking advantage of this fact.

Accordingly, in an embodiment, the present invention relates to unexpectedly high quality production of pupae/insects. The quality control of the insects processing line can be enhanced with the knowledge that pre-treatment steps can be employed. For example, in one pre-treatment step, larvae can be chilled before employing a killing/deactivation step. Thus, the processing of insects can involve freeze-drying (lyophilization). Testing has confirmed that ultra-low temperatures are fine, and the present invention contemplates potentially using those temperatures. However, special and more expensive cooling equipment might be necessary for those temperatures. Thus, the present invention contemplates taking advantage of the cooling of the pupae/insects but without having to resort to special more expensive equipment. For example, the present invention contemplates performing processing insects in the 0 to −10° C., or 0 to −20° C. or −10 to −20° C. range.

The present invention also contemplates doing production at elevated temperatures or with other processing steps. For example, it is contemplated that the knowledge that dead insects fluoresce can be used to one's advantage by employing fluorescence after drying larvae, or grinding, or after fat extraction. It has been confirmed by experimental testing that at high temperatures, fluorescence still occurs, thus, the present invention contemplates being able to employ techniques such as boiling water (for example for sterilization purposes), drying the insects/pupae with hot air, and/or microwaving them. Testing has confirmed that drying diminishes the fluorescent effect to a small degree, but it is postulated that the effect may be linked with lowering humidity rather than using the elevated temperature. In an embodiment, the present invention contemplates using fluorescence after drying larvae with the hot air, or blanching with boiling water or drying them with microwaves.

Experimental testing has confirmed that an amount of dead insects can be estimated after undergoing the drying process, regardless of the type of drying process used. For example, hot air, microwave, and/or freeze-drying have all been shown to work. In one variation, it is contemplated that drying can be utilized without greatly adversely affecting the fluorescent quality. This can be used advantageously as a quality control measure or even to allow simple separation. The ultimate result is that it will allow a better final product to be achieved.

It has been confirmed by experimental testing that water used for blanching insects is a perfect indicator of how many dead/sick individuals were in the production batch (unlike larvae intentionally deactivated and processed). The fluorescence intensity present in the water that was in contact with insects shows the fluorescence phenomenon with the intensity being directly proportional to the number of sick and/or dead insects. The fluorescence level (and Beer's law) allows one to calculate the number and a standard curve can likely be used to detect the number of sick and/or dead insects.

It has also been confirmed that even after employing further steps of insect processing does not greatly adversely affect the luminescence of the pupae/insects, thereby allowing evaluation of the number of dead individuals in any batch, even in final insect meal (insects' powder).

Although fluorescence can be used as a measure after a plurality of processing or production steps, the inventors recognize that fluorescence does become weaker and eventually fades away over time. Thus, in one embodiment, the inventors propose using a standardized curve over time to ascertain the diminishment of the fluorescent intensity, thereby allowing the technique to be used even when the fluorescent intensity is diminished. This also means that a quality check may be possible in the products that are stored for several days and longer. Without being bound by theory, the inventors contemplate that low temperatures might extend the fluorescent time, which may potentially be used advantageously to allow insect broods to be stored longer.

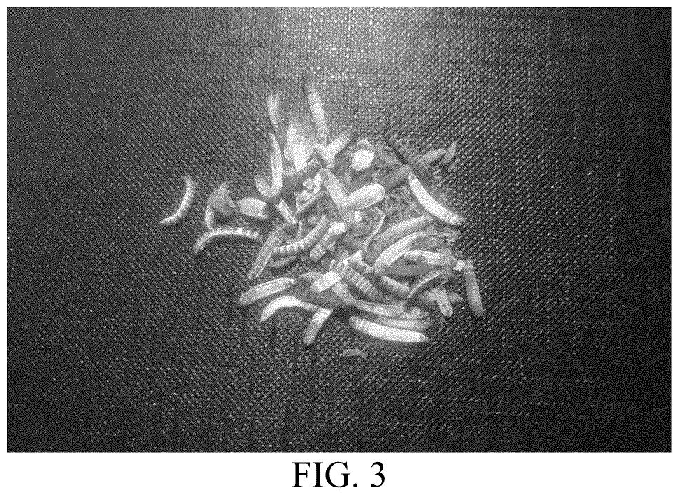

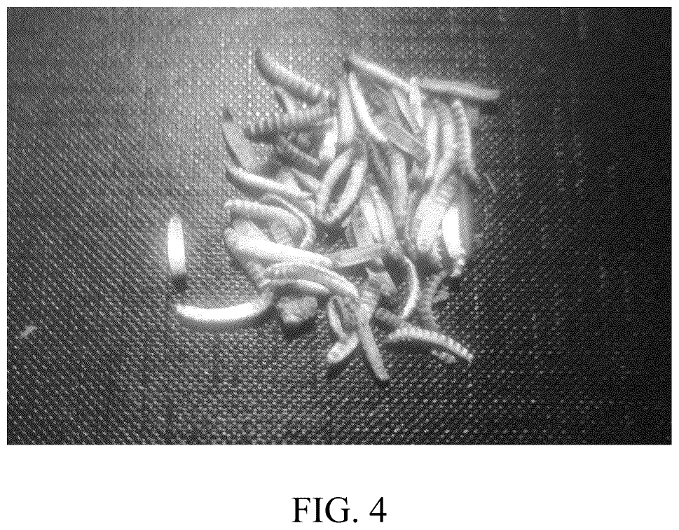

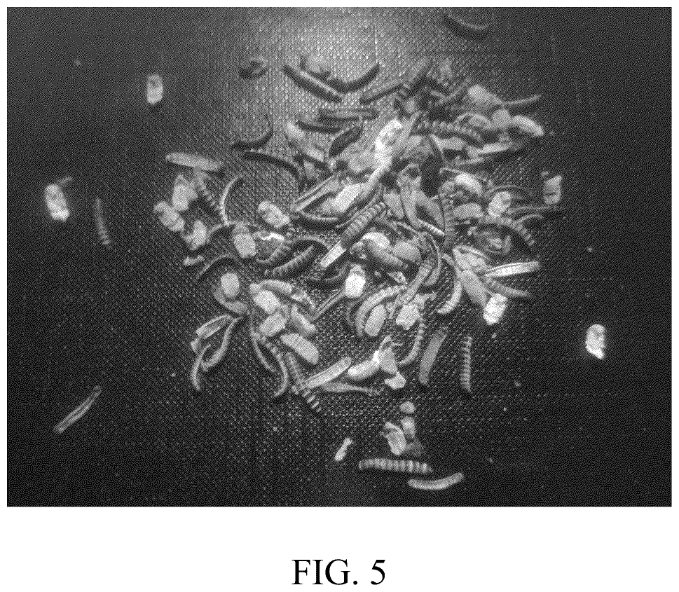

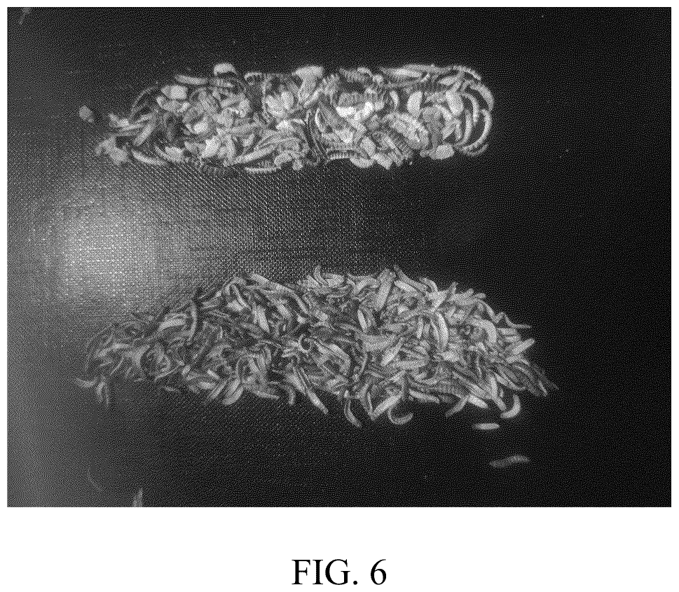

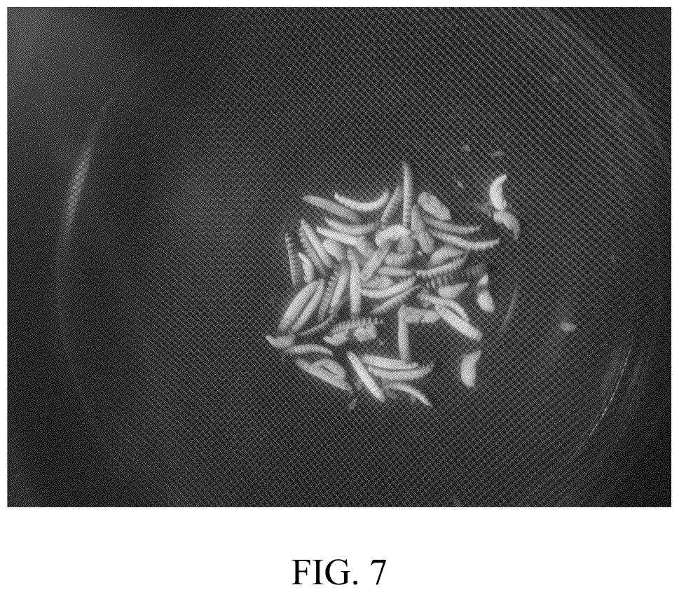

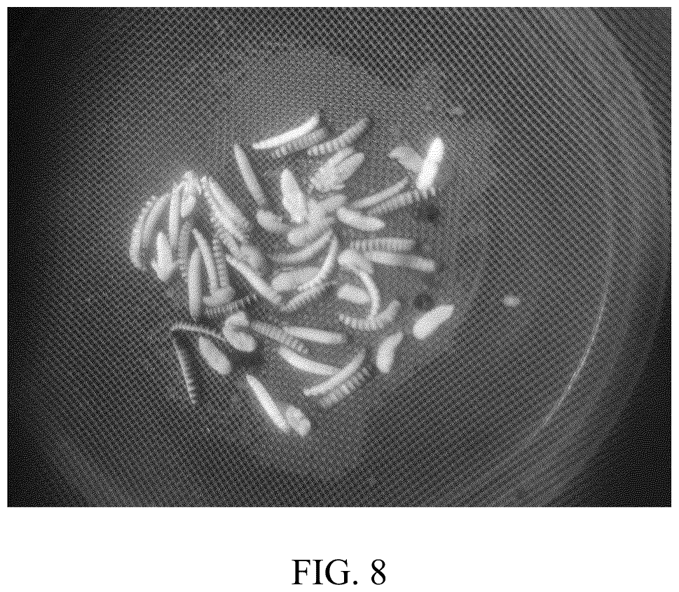

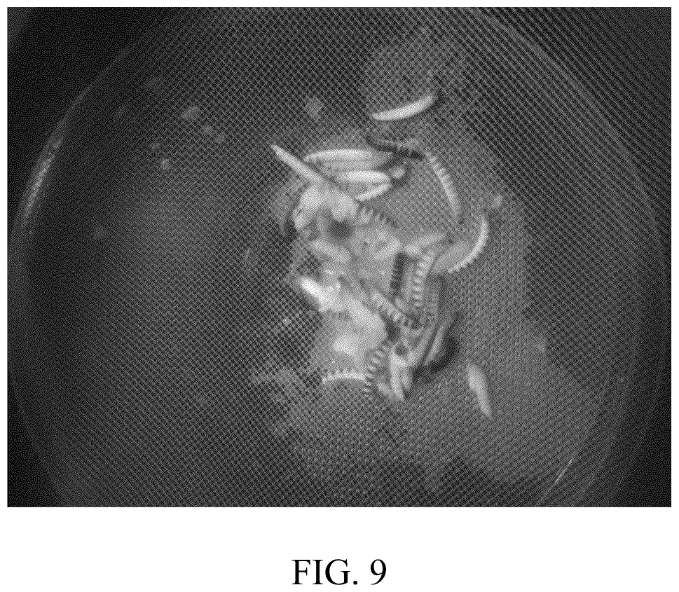

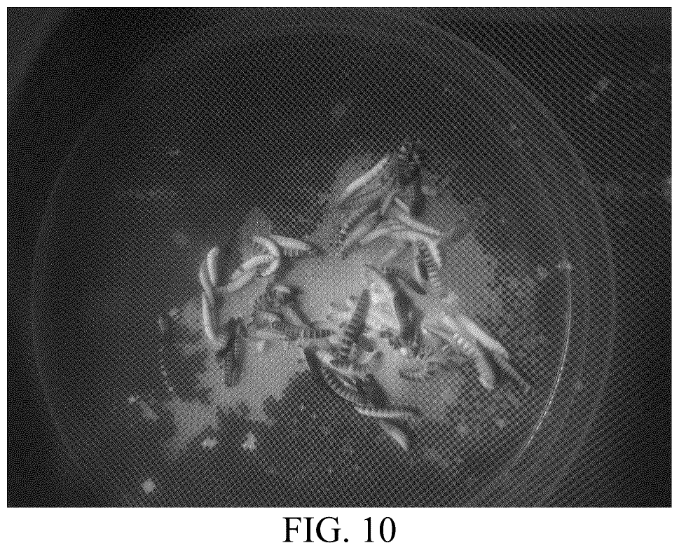

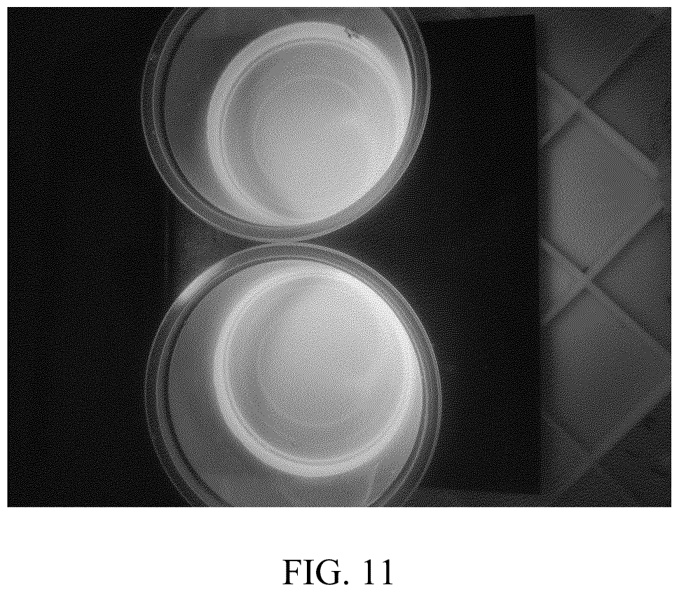

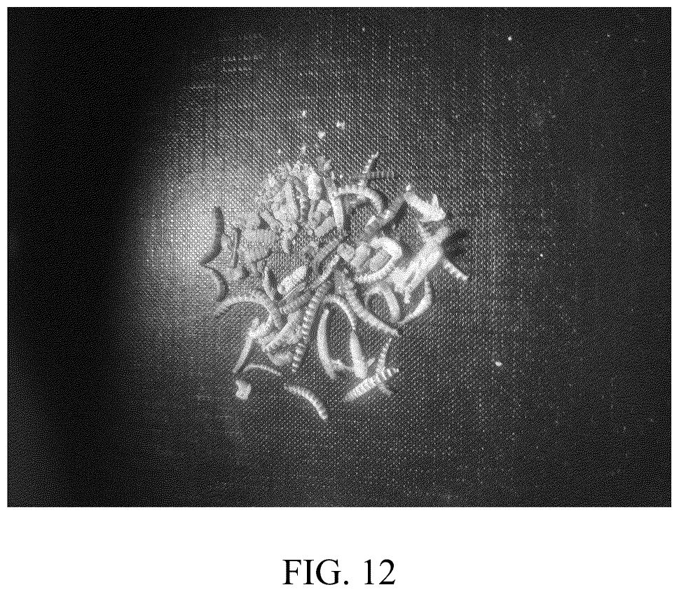

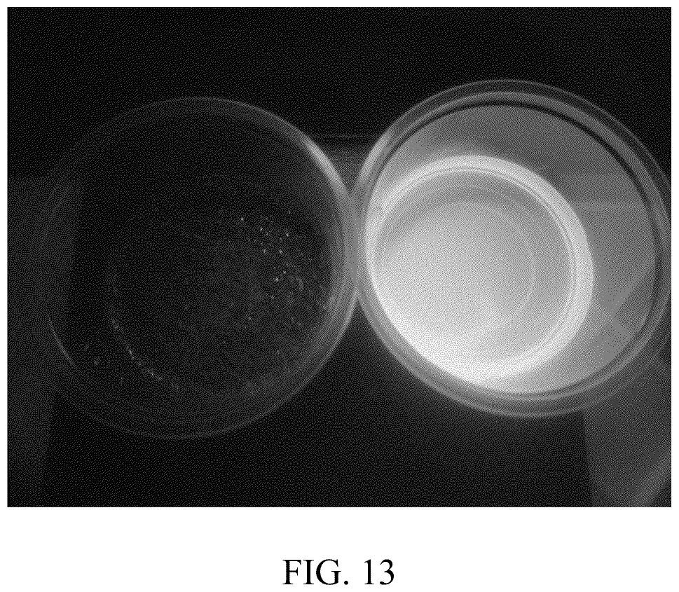

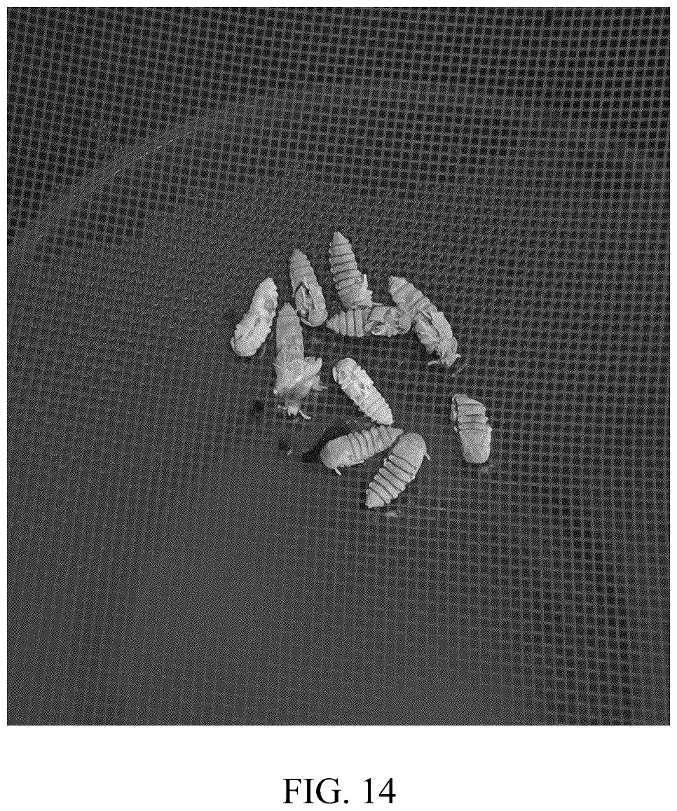

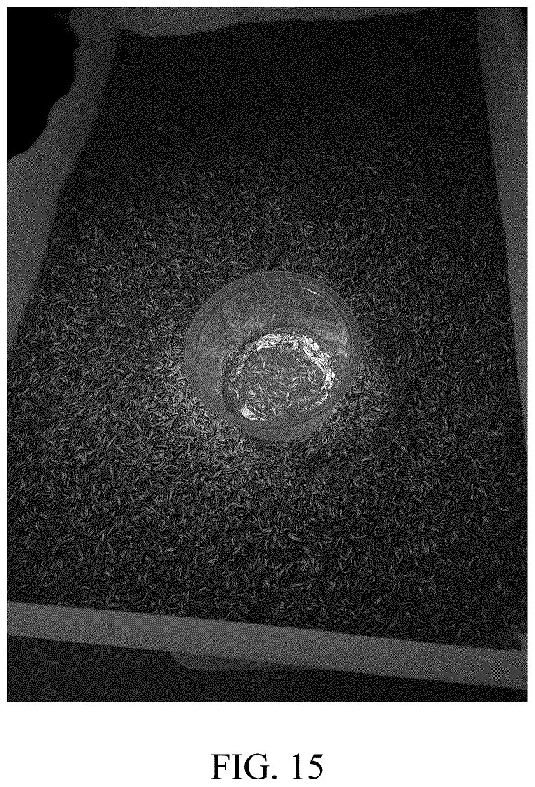

FIG. 3-23B show the effects of various parameters, such as cold, heat, air, and humidity as a function of time on both the larvae and/or pupae as well as the liquid derived from the larvae and/or pupae as it relates to the larvae and/or pupae and their fluorescent effect (and intensity) after treatment with irradiation (generally at 395 nm with a flashlight). FIG. 3 shows dead larvae and pupae after a night in the fridge at 7° C. It can be seen that there is no visible impact on the fluorescent effect. FIG. 4 shows dead larvae and pupae after a night in the freezer at −20° C., wherein there is no visible impact on the fluorescent effect. FIG. 5 shows dead larvae and pupae after drying in 80° C. after 3 h, wherein the fluorescent effect is a bit weaker, but nevertheless, still present. Thus, it appears that heat over a period of time appears to diminish the fluorescent effect. FIG. 6 shows dead larvae and pupae from FIG. 5 with fluorescent effect in the top image whereas the dead larvae and pupae that were deactivated and dried (underwent a processing step) with a hot air in the bottom of the image showed no appreciable fluorescent effect. This indicates that a processing step can take place without adversely affecting the fluorescent effect. FIG. 7 shows dead larvae and pupae before pouring boiling water on to them and FIG. 8 shows dead larvae and pupae after pouring boiling water on them. This shows that boiling the larvae and pupae has no visible impact on the fluorescent effect. FIG. 9 shows dead larvae and pupae after pouring boiling water and leaving them for a night and the fluorescent effect is still strong. Thus, even after some time, the fluorescent effect is still present. FIG. 10 shows dead larvae and pupae after pouring cold water and leaving for a night. These larvae and pupae also demonstrated a fluorescent effect. FIG. 11 shows a comparison of water from larvae and pupae bath wherein the top image shows them after boiling, and the bottom image shows them after being exposed to cold. Both showed a strong fluorescent effect indicating that neither heat nor cold diminishes the fluorescent effect to any great extent. FIG. 12 shows larvae and pupae after drying in a microwave for 10 min and the fluorescent effect is weaker, but still present. FIG. 13 shows on the right, water from a larvae and pupae bath (that came from the larvae and pupae in FIG. 11) and on the left, it shows water with larvae that was intentionally deactivated (killed) and dried. These larvae and pupae demonstrated no fluorescent effect. FIG. 14 shows the detail on pupae up close wherein one can see the segmentation in the body parts. FIG. 15 shows larvae and pupae in a cup wherein there is a fluorescent effect present whereas in the background, a large amount of larvae are shown after deactivation and drying with no fluorescent effect.

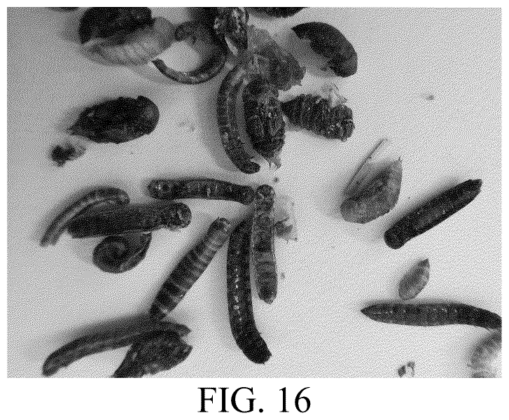

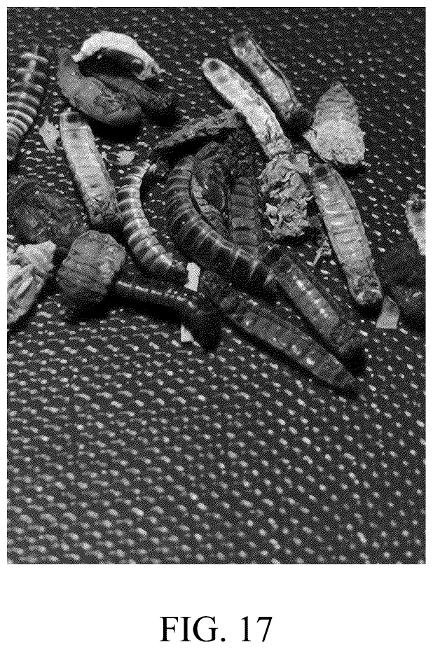

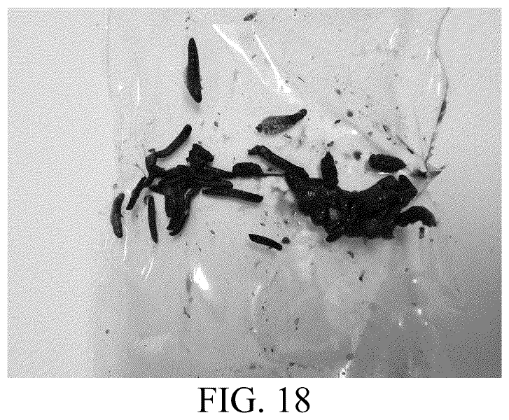

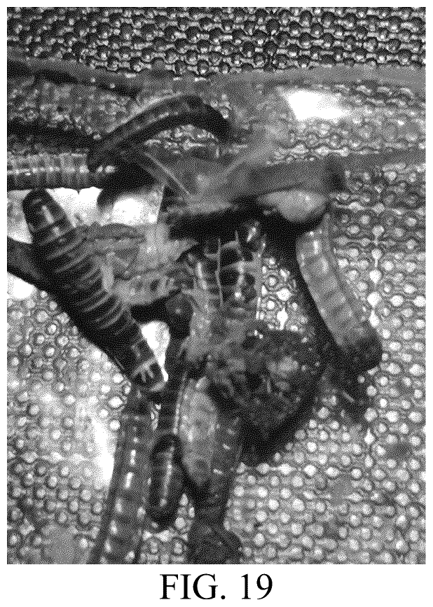

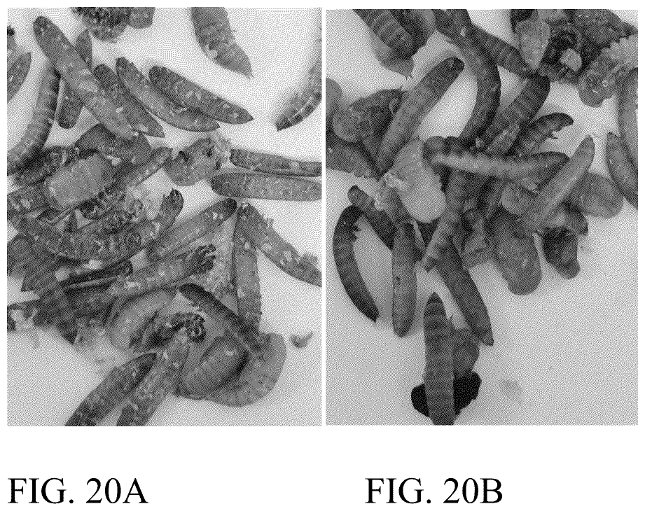

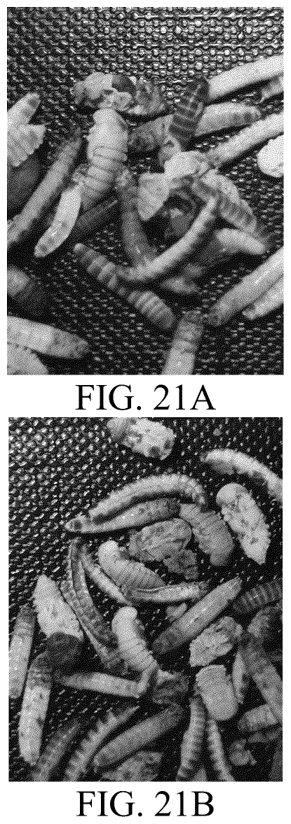

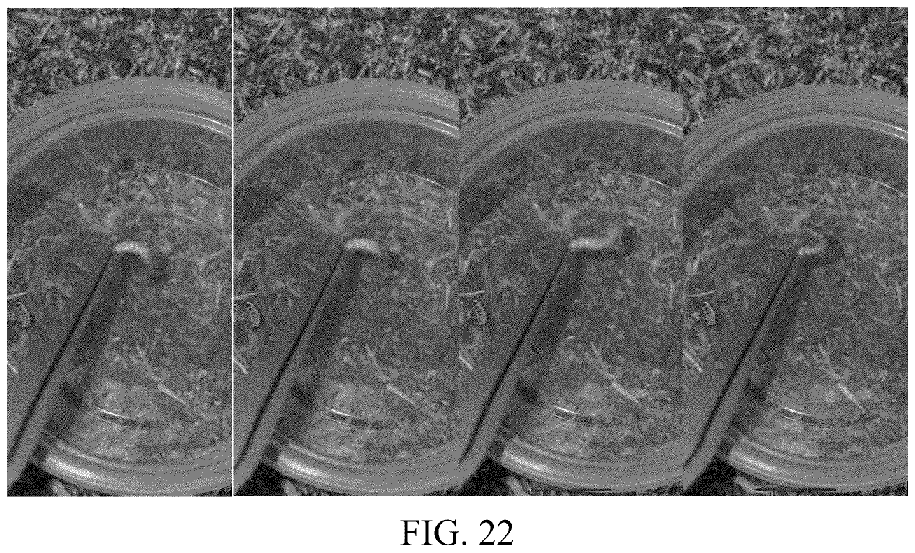

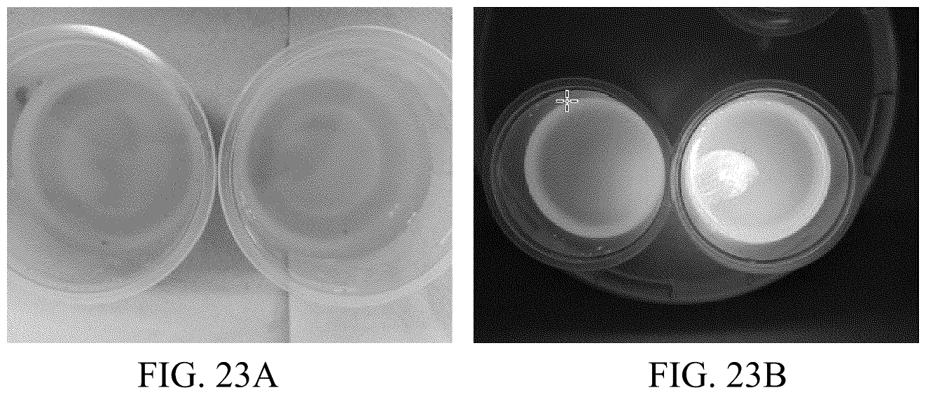

In the presence of air at 6° C. for two weeks, the experiment showed pupae that were dry and darker when exposed to white light (see FIG. 16). FIG. 17 shows that there is still a fluorescent effect (but slightly diminished) after 2 weeks at 6° C. The lack of humidity may diminish the effect. Other experiments demonstrated that in the absence of air, the decomposition process occurs wherein the larvae and/or pupae turn black and begin attaining a goo-like consistency (see FIG. 18). Even after two weeks under refrigerated conditions (6° or 7° C.), fluorescence occurs (see FIG. 19). The larvae and/or pupae show little difference after two weeks under refrigerated conditions at −20° C. from the larvae and/or pupae immediately after being bred under white light (see FIGS. 20A and 20B). Similarly, both groups showed similar levels of fluorescence after irradiation (see FIGS. 21A and 21B). FIG. 22 shows the results of a sick insect that is in a terminally ill condition that is about to die, wherein the presence of fluorescence gives this indication. The image FIG. 22 shows a time lapse sequence over a period of about 1 second. Finally, FIGS. 23A and 23B show the results of liquid from larvae and pupae measured at time 0 (left side) and at a time 2 weeks later (right side) wherein the larvae and pupae were stored at 6° C. Both the white light results (23A) and the fluorescent results (23B) were similar at time 0 and at two weeks' time.

In an embodiment, the present invention relates to a system for detecting one or more dead insects in a pool of insects, said system comprising a digital circuit, a UV and/or visible light source, one or more photodetectors, and a conveyor, the UV and/or visible light source and the one or more photodetectors, wherein the UV and/or visible light source transmits pulses of UV and/or visible light at the pool of insects, the pool of insects present on the conveyor that moves the pool of insects, wherein if one or more dead insects is present, the one or more dead insects transmits light that is detected by the one or more photodetectors triggering a response in the one or more photodetectors that alerts the user to a presence of the one or more dead insects.

In a variation, the computer can be connected operationally to the conveyor and comprises a computer program that allows the computer to determine a precise location of the one or more sick or dead insects.

In a variation, the peak intensity of the electromagnetic radiation emitted by the insects are at their highest intensity shortly after death and as time passes, the intensity decreases. That is, the intensity of the electromagnetic radiation emitted goes up after the insect dies until it reaches a peak intensity. After attaining its peak intensity, the intensity diminishes until the intensity of fluorescent light emitted gets to a point where it is no different compared to the living insects. Accordingly, in one embodiment, the present invention relates to being able to ascertain an exact time of death, as there is a dependence between the intensity of the light emitted and the amount of time from death. In an embodiment, a standard curve can be prepared that relates intensity of the emitted light relative to the time after death of the one or more insects. In an embodiment, the insects should be sampled at time intervals that allow one to ascertain when the intensity of light of the one or more sick or dead insects is increasing and when it is decreasing.

In a variation, the pulses of UV and/or visible light occur at time intervals of 1 millisecond to 100 milliseconds. In a variation, ten percent of the pool of insects is sampled with each pulse of UV and/or visible light.

In an embodiment, the one or more photodetectors comprise one or more of a band pass filter and a lens. In a variation, the one or more photodetectors comprise both the band pass filter and the lens. In a variation, the band pass filter allows light of 475 to 500 nm to pass.

In a variation, the computer instructs the conveyor to move at a pace and a direction that allows 100 percent of the pool of insects to be sampled at an interval of between one minute and one hour. In a variation, the pool of insects are one or more members selected from the group consisting of Coleoptera, Tenebrio molitor, Alphitobius diaperinuis, and Zophobas morio.

In a variation, the one or more photodetectors are operationally attached to LEDs. In a variation, the one or more photodetectors are positioned in a row.

In a variation, the conveyor moves the insects in a circular direction or in a side to side direction.

In an embodiment, the present invention relates to a method of detecting sick and/or dead insects in a pool of insects, the method comprising:
a) placing the pool of insects on a conveyor that moves the insects,
b) irradiating at least a portion of the pool of insects on the conveyor with a pulse of UV and/or visible light,
wherein any sick and/or dead insects in the portion of the pool of insects emit a light that is detected by one or more photodetectors,
c) employing a computer that comprises a computer program that operationally connects the pulse of UV and/or visible light to a movement of the insects on the conveyor, and thereby allows a user to detect the sick and/or dead insects.

In a variation of the method, the method may further comprise separating the insects from feed and/or frass prior to placing the pool of insects on the conveyor.

In a variation of the method, the computer program alerts the user to a precise location of the sick and/or dead insects. In a variation, the one or more photodetectors comprise a lens and a band pass filter. In a variation, the pool of insects are one or more members selected from the group consisting of Coleoptera, Tenebrio molitor, Alphitobius diaperimis, and Zophobas morio.

In a variation of the method, the band pass filter allows light of 435 to 465 nm to pass.

In a variation, the portion of the pool of insects is between about one tenth and one fifth of the total pool of insects. In a variation, a time interval for each pulse of UV and/or visible light is between one second and 10 minutes. In a variation, the conveyor moves the pool of insects in a circular direction or in a side-to-side direction.

In an embodiment, the methods of the present invention can include a step during growth that facilitates the ability to get a better product. This step may include heating, cooling, drying, and/or adding humidity. The results shown herein demonstrate that the dead pupae and/or larvae will still show significant fluorescent activity even after exposing the pupae and/or larvae to conditions that differ from room temperature and a regular humidity. Moreover, without being bound by theory, the use of cold may extend the fluorescent activity so that a cooling step may be beneficial. Any of the change in conditions enumerated herein may be performed as a preliminary step in order to get better yield and/or purer or better product and generally can be used to more readily identify dead insects so that they can be more rapidly and efficiently removed.

In a variation, the present invention also relates to a step in a method or an element in a system wherein one or more filters are used to allow one to better differentiate between dead and living healthy pupae and/or larvae, due to being able to more readily differentiate between the sick/dead insects that are undergoing luminescence (fluorescence) from the healthy live insects that may be reflecting incident light. The difference in the incident radiation and the emitted radiation allow these one or more filters to be useful.

In an embodiment, the present invention relates to a method of detecting a number of sick and/or dead insects in a pool of insects, the pool of insects comprising water, said method comprising:
a) procuring the water from the pool of insects,
b) irradiating the water with a pulse of UV and/or visible light,
c) measuring a fluorescent intensity of the water that has been subjected to the pulse of UV and/or visible light,
d) employing any one of a computer, Beer's law, or a standard curve to calculate the number of sick and/or dead insects from the fluorescent intensity.

In a variation, the method can be used to calculate a percentage or ratio of sick and/or dead insects. In a variation, the method further comprises using fragments of sick and/or dead insects, and the method allows detection of the fluorescent intensity using a pool of crushed and/or ground individuals. In a variation, the method can be used before, during, or after processing steps occur. For example, the sick and/or dead insects and/or fragments thereof may be detected in a processing step wherein fat pressing is used (separating liquids from solids by the application of high mechanical pressure). In a variation, the sick and/or dead insects and/or fragments thereof or the mother liquid thereof may allow detection in defatted insect meal. In a variation, the sick and/or dead insects and/or fragments thereof may be detected in insect chitin.

In an embodiment, the present invention relates to adding a known aliquot size of water or an appropriate solvent to the pool of insects. Appropriate solvents are solvents that can extract the luminescent (fluorescent) compound from the insects that will not interfere with the luminescent (fluorescent) properties of the extracted luminescent (fluorescent) compound. For example, solvents such as C₁₋₅ alcohols (e.g. ethanol, propanol, isopropanol, butanol, etc.), ethers, acetone, ethyl acetate, glycols, chlorinated hydrocarbons (such as methylene chloride or chloroform), benzene, toluene are contemplated as potential appropriate solvents. Because the size of the aliquot of the solvent added to the pool of insects is known, one can use the relative emission of the fluorescent intensity that appears in the water or appropriate solvent after subjecting the water or appropriate solvent to the incident light to cause the fluorescence from the compound. Using Beer's Law, standard curves, and/or computers will allow one to calculate the relative number of sick and/or dead insects in that pool of insects (i.e., a concentration of the fluorescent compound can be determined, and an estimate of the number of dead and/or sick insects can be made). In a variation, it should be understood that the extraction of the fluorescent compound may be improved if the insects are ground and/or crushed (the extraction process will likely proceed more rapidly).

It should be understood and it is contemplated and within the scope of the present invention that any feature that is enumerated above can be combined with any other feature that is enumerated above as long as those features are not incompatible. Whenever ranges are mentioned, any real number that fits within the range of that range is contemplated as an endpoint to generate subranges. In any event, the invention is defined by the below claims.

We claim:

1. A system for detecting one or more sick and/or dead insects in a pool of insects, said system comprising a digital circuit, a UV and/or visible light source, one or more photodetectors, and a conveyor, the digital circuit being operationally attached to the UV and/or visible light source and the one or more photodetectors, wherein the UV and/or visible light source transmits pulses of UV and/or visible light at the pool of insects, the pool of insects present on the conveyor that moves the pool of insects, wherein if one or more sick and/or dead insects is present, the one or more sick and/or dead insects transmits light that is detected by the one or more photodetectors triggering a response in the one or more photodetectors that alerts the user to a presence of the one or more sick and/or dead insects.

2. The system of claim 1, wherein a precise time of death can be detected for the one or more dead insects.

3. The system of claim 1, wherein the system optionally further comprises a computer, the computer comprises a computer program that provides/processes additional data.

4. The system of claim 1, wherein the pulses of UV and/or visible light occur at time intervals of 1 millisecond to 10 minutes, allowing the system to perform continuous inspection in real time.

5. The system of claim 3, wherein ten percent or less of the pool of insects is sampled with each pulse of UV and/or visible light.

6. The system of claim 1, wherein the one or more photodetectors comprise one or more of a band pass filter and a lens, and optionally wherein the band pass filter allows light of 460 to 490 nm to pass.

7. The system of claim 1, wherein the computer instructs the conveyor to move at a pace and a direction that allows 100 percent of the pool of insects to be sampled at an interval of between one millisecond and 100 milliseconds.

8. The system of claim 1, wherein the pool of insects are of the Coleoptera order or are one or more species selected from the group consisting of Alphitobius diaperimuis, Alphitobius laevigatus, Tenebrio molitor, Tenebrio obscurus, Tenebrio opacus, Zophobas atratus, and Zophobas morio, which are at a same or different stage of development.

9. The system of claim 1, wherein the one or more photodetectors are operationally attached to LEDs, and are optionally positioned in a row.

10. The system of claim 1, wherein the conveyor moves the insects in a circular direction or in a side-to-side direction.

11. A method of detecting sick and/or dead insects in a pool of insects, said method comprising:
a) placing the pool of insects on a conveyor that moves the pool of insects,
b) irradiating a portion of the pool of insects on the conveyor with a pulse of UV and/or visible light,
wherein any sick and/or dead insects in the portion of the pool of insects transmit a light that is detected by one or more photodetectors,
c) optionally employing a computer that comprises a computer program that operationally connects the pulse of UV and/or visible light to a movement of the insects on the conveyor, and thereby allows a user to detect the sick and/or dead insects.

12. The method of claim 11, wherein the computer program alerts the user to a precise location of the sick and/or dead insects.

13. The method of claim 11, wherein the one or more photodetectors comprise a lens and a band pass filter.

14. The method of claim 11, wherein the pool of insects are one or more members selected from the group consisting of Coleoptera, Alphitobius diaperimis, Alphitobius laevigatus, Tenebrio molitor, Tenebrio obscurus, Tenebrio opacus, Zophobas atratus, and Zophobas morio, wherein the one or more members are at different stages of development.

15. The method of claim 11, wherein the band pass filter allows light of 460 to 490 nm to pass.

16. The method of claim 15, wherein the portion of the pool of insects is between about one tenth and one fifth of the total pool of insects, and optionally, wherein a time interval for each pulse of UV and/or visible light is between one millisecond and 100 milliseconds.

17. The method of claim 11, wherein the conveyor moves the pool of insects in a circular direction or in a side-to-side direction.

18. A method of detecting a number of sick and/or dead insects in a pool of insects, the pool of insects comprising water or an appropriate solvent, said method comprising:
a) procuring the water or the appropriate solvent from the pool of insects,
b) irradiating the water or the appropriate solvent with a pulse of UV and/or visible light,
c) measuring a fluorescent intensity of the water or the appropriate solvent that has been subjected to the pulse of UV and/or visible light,
d) employing any one of a computer, Beer's law, or a standard curve to calculate the number of sick and/or dead insects from the fluorescent intensity.

19. The method of claim 18, further comprising calculating a percentage or ratio of sick and/or dead insects.

20. The method of claim 18, wherein the water of the appropriate solvent comes from water or the appropriate solvent added to the pool of insects, wherein the appropriate solvent is ethanol.
